(19) United States
(12) Patent Application Publication
(10) Pub. No.: US 2014/0039443 A1
(43) Pub. Date: Feb. 6, 2014

(54) ABSORBENT ARTICLE

(71) Applicants: Takahito Nagai, Kagawa (JP); Tomoko Tsuji, Kagawa (JP); Nobuhiro Tagawa, Kagawa (JP)

(72) Inventors: Takahito Nagai, Kagawa (JP); Tomoko Tsuji, Kagawa (JP); Nobuhiro Tagawa, Kagawa (JP)

(73) Assignee: UNICHARM CORPORATION, Shikokuchuo-shi, Ehime (JP)

(21) Appl. No.: 13/982,963

(22) PCT Filed: Feb. 1, 2012
(86) PCT No.: PCT/JP2012/052214

§ 371 Date: Oct. 11, 2013

(30) Foreign Application Priority Data
Feb. 1, 2011 (JP) 2011-020259

(51) Int. Cl. A61F 13/58

(52) U.S. Cl. CPC A61F 13/58
USPC 604/389

(57) ABSTRACT

Provided is an absorbent article such as a disposable diaper wherein, when using a configuration that attaches the fastening tapes directly to the outer nonwoven fabric layer without using target tapes, the strength of the outer nonwoven fabric layer can be maintained while the fastening strength of the fastening tapes is maintained even with repeated use. A disposable diaper (1) is provided with an adhesive layer (80) that is formed from an adhesive and glues a barrier sheet (20) to an outer-layer nonwoven fabric layer (40). The adhesive layer (80) has an adhesive-coating pattern such that the orientation of the fibers forming the outer-layer nonwoven fabric layer (40) and the orientation of the resin forming the barrier sheet (20) intersect.

### TECHNICAL FIELD

The present invention relates to an absorbent article such as an open-type disposable diaper using a fastening tape.

### BACKGROUND ART

Conventionally, there is known an open-type disposable diaper using a fastening tape. Such a disposable diaper often uses, as the fastening tape, a structure in which a male member of a mechanical fastener is provided on a side panel extending from the dorsal region of the disposable diaper. Furthermore, in the ventral region of such a disposable diaper, generally, a target tape that engages with the male member of the mechanical fastener is provided.

For such a disposable diaper, there is proposed a disposable diaper in which rather than using the target tape, that is, the female member of the mechanical fastener, the male member of the mechanical fastener is directly engaged with an outer-layer nonwoven fabric arranged outside an barrier sheet of the disposable diaper (for example, Patent Literature 1). In the disposable diaper, in order to ensure a perfect engagement of the male member with the outer-layer nonwoven fabric, as well as the strength of the outer-layer nonwoven fabric, adhesive-applied units of the barrier sheet and the outer-layer nonwoven fabric are provided linearly at a predetermined interval.

### CITATION LIST

### Patent Literature

### [Patent Literature 1]

Japanese Patent Application Publication No. 2001-46426 (Page 3, FIG. 2)

### SUMMARY OF INVENTION

However, the aforementioned conventional disposable diaper has the problems below. That is, the adhesive-applied units of the barrier sheet and the outer-layer nonwoven fabric are linearly provided, and therefore, the adhesive easily seeps out to the surface of the outer-layer nonwoven fabric. The adhesive that seeps out restricts the free movement of the fibers forming the outer-layer nonwoven fabric, and thus, the engagement force of the male member of the mechanical fastener with the outer-layer nonwoven fabric declines.

Furthermore, in a portion of the outer-layer nonwoven fabric that is not bonded with the barrier sheet, the repeated use of the fastening tape stretches and keeps undoing the fibers engaged with the male member, and as a result, the outer surface of the outer-layer nonwoven fabric is easily covered. This further declines the engagement force of the male member with the outer-layer nonwoven fabric.

Thus, the present invention has been achieved in view of such a situation, and an object thereof is to provide an absorbent article, such as a disposable diaper, by which it is possible to maintain the engagement force of the fastening tape even after repeated use while ensuring the strength of an outer-layer nonwoven fabric, in a case of using a structure in which rather than making use of a target tape, the fastening tape is engaged directly with the outer-layer nonwoven fabric.

A feature of the present invention is summarized as an absorbent article (disposal diaper 1) comprising: a liquid-permeable topsheet (topsheet 10), a liquid-impermeable barrier sheet (barrier sheet 20), a liquid-retentive absorber (absorber 30) provided between the topsheet and the barrier sheet, and an outer-layer nonwoven fabric (outer-layer nonwoven fabric 40) provided outside the barrier sheet, comprising, a substantially longitudinally-elongated absorbent chassis (absorbent chassis 50); side panels (side panels 60) extending from sides of the absorbent chassis; a male member (male member 71a, male member 72a) of a mechanical fastener provided at sides of the side panels; and an adhesive layer (adhesive layer 80) arranged between the barrier sheet and the outer-layer nonwoven fabric, and formed by an adhesive (adhesive 81) that bonds the barrier sheet and the outer-layer nonwoven fabric, wherein the adhesive layer has an application pattern of the adhesive, the application pattern crossing an orientation of fibers forming the outer-layer nonwoven fabric and an orientation of a resin forming the barrier sheet.

### BRIEF DESCRIPTION OF DRAWINGS

FIG. 1 is an exploded plan view of a disposable diaper 1 according to an embodiment of the present invention.

FIG. 2 is a diagram schematically illustrating a cross section of the disposable diaper 1 along F2-F2 shown in FIG. 1.

FIG. 3 is a diagram illustrating an application pattern of an adhesive in an adhesive layer 80 according to an embodiment of the present invention.

FIG. 4 is a diagram illustrating an application pattern of an adhesive in an additional adhesive layer 90 according to an embodiment of the present invention.

### DESCRIPTION OF EMBODIMENTS

Next, an embodiment of an absorbent article according to the present invention is explained with reference to drawings. In the following description of the drawings, the same or similar reference numerals are used to designate the same or similar parts. It will be appreciated that the drawings are schematically shown and the ratio of each dimension, for example, are different from the real ones.

Therefore, a specific dimension should be determined in view of the following description. Moreover, among the drawings, the respective dimensional relations or ratios may differ.

### (1) Overall Schematic Configuration of Absorbent Article

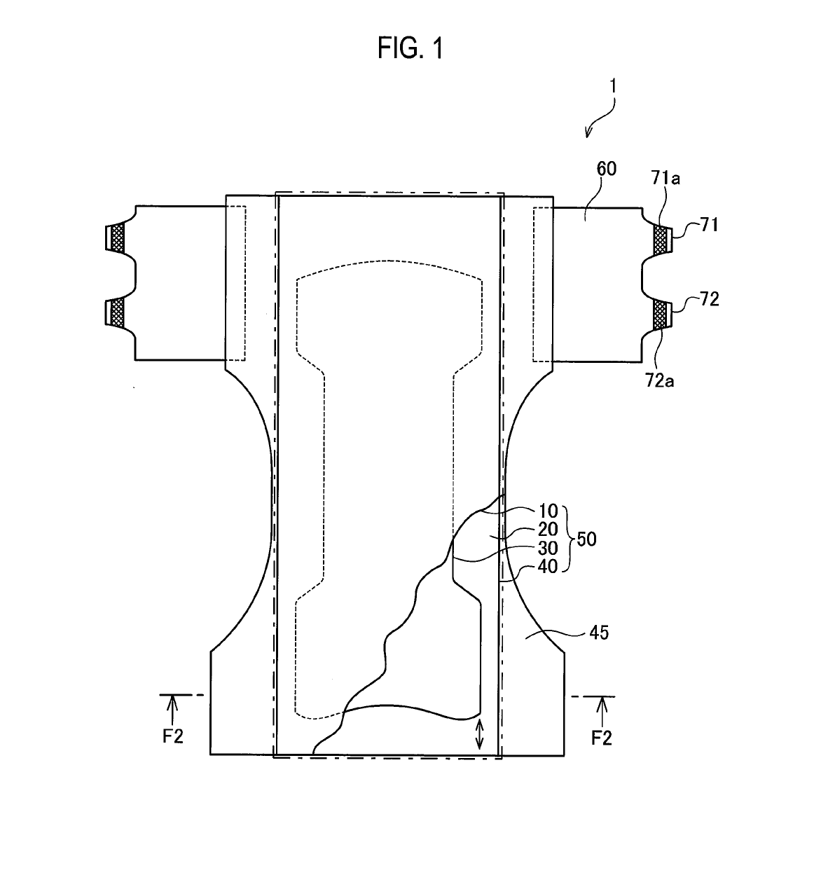

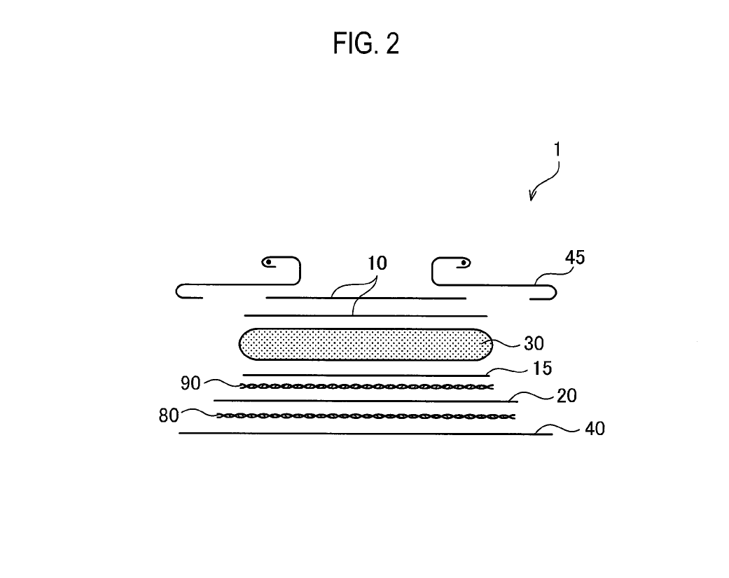

FIG. 1 is an exploded plan view of a disposable diaper 1, which is an embodiment of the absorbent article according to the present invention. FIG. 2 is a diagram schematically illustrating a cross section of the disposable diaper 1 along F2-F2 shown in FIG. 1.

As illustrated in FIG. 1 and FIG. 2, the disposable diaper 1 is provided with a topsheet 10, a barrier sheet 20, an absorber 30, and an outer-layer nonwoven fabric 40, and includes a substantially longitudinally-elongated absorbent chassis 50.

The topsheet 10 is a liquid-permeable sheet that is in direct contact with the skin of a wearer. The topsheet 10, for example, is formed by a sheet made of hydrophilic nonwoven fabric, an aperture plastic film, or an aperture hydrophobic nonwoven fabric.

The barrier sheet 20 is a liquid-impermeable sheet that prevents the liquid absorbed in the absorber 30 from leaking to the outside. The barrier sheet 20, for example, is formed by a polyethylene (PE) film sheet.

The absorber 30, which has a longitudinally elongated shape, has liquid retention property that the liquid excreted from the wearer is absorbed. The absorber 30, for example, is formed by a material obtained by mixing cotton-like pulp and a highly polymerized absorbent polymer (SAP). The absorber 30 is provided between the topsheet 10 and the barrier sheet 20.

It should be noted that in the present embodiment, a lower-layer sheet 15 (see FIG. 2) is further provided between the barrier sheet 20 and the absorber 30. The lower-layer sheet 15 is a sheet narrower in width than the barrier sheet 20. The lower-layer sheet 15, for example, is formed by a tissue or a hydrophilic nonwoven fabric sheet.

The outer-layer nonwoven fabric 40 is provided outside the barrier sheet 20. The material used as the outer-layer nonwoven fabric 40 is not particularly limited as long as fastening tapes 71, 72 provided in side panels 60 can be engaged; however, examples of preferred constituent fibers include fibers that consists only of polyolefin fibers such as polyethylene (PE) fibers and polypropylene (PP) fibers, and thermoplastic resins such as polyethylene terephthalate (PET) and polyamide. Another example thereof may include composite fibers having a core-sheath type or a side-by-side type structure, that is, fibers having a core-sheath structure in which the core component is a PE or a low-melting point PP. In addition, the outer-layer nonwoven fabric 40 may be composed by using these fibers either individually or in a combination of two or more types.

On both sides of the outer-layer nonwoven fabric 40, a leg side gather (LSG) nonwoven fabric 45 having elastic members such as elongated synthetic rubber arranged along the longitudinal direction of the absorbent chassis 50 is provided.

A pair of left-right side panels 60 extending from the sides of the absorbent chassis 50 are bonded between the outer-layer nonwoven fabric 40 and an LSG nonwoven fabric 45. The side panels 60 are formed to be capable of expanding and contrasting in a shorter direction perpendicular to the longitudinal direction of the absorbent chassis 50. A well-known method can be used as a method of providing such flexibility to the side panels 60, and examples thereof may include a method of bonding a plurality of elastic members that are elongated in the shorter direction onto a nonwoven fabric forming the side panels 60.

The fastening tape 71 and the fastening tape 72 are provided at the sides of a pair of left-right side panels 60. A male member 71a of the mechanical fastener is provided on the fastening tape 71. Similarly, a male member 72a of the mechanical fastener is provided on the fastening tape 72.

The pair of left-right side panels 60 are provided in a dorsal region of the absorbent chassis 50 positioned at the dorsal side of the wearer. In the disposable diaper 1, a target tape (a female member of the mechanical fastener) where the male member 71a and the male member 72a are engaged is not provided. That is, the male member 71a and the male member 72a are engaged directly with the surface of the outer-layer nonwoven fabric 40. Specifically, the male member 71a and the male member 72a can be engaged to any region (within a frame indicated by a dashed line in FIG. 1) on the surface of the outer-layer nonwoven fabric 40.

It should be noted that such a structure of the disposable diaper 1 is same as that of a well-known open-type disposable diaper.

The present embodiment is designed such that in a state where the side panels 60 are elongated to the maximum extent and the male member 71a and the male member 72a are engaged with the outer-layer nonwoven fabric 40, the stress exerted on the side panels 60 is 4.0 N or less. Furthermore, two, not one, male members are provided on each of the side panels 60, and therefore, the stress is distributed by the elastic members of the side panels 60 so as to achieve a feature that the stress obtained when the side panels 60 are elongated to the maximum extent is 4.0 N or less. A method of measuring the stress will be described later.

Furthermore, as illustrated in FIG. 2, between the barrier sheet 20 and the outer-layer nonwoven fabric 40, an adhesive layer 80 formed by an adhesive that bonds the barrier sheet 20 and the outer-layer nonwoven fabric 40 is provided. The adhesive layer 80 bonds the barrier sheet 20 and the outer-layer nonwoven fabric 40.

Moreover, in the present embodiment, between the barrier sheet 20 and the lower-layer sheet 15, an additional adhesive layer 90 formed by an adhesive that bonds the barrier sheet 20 and the lower-layer sheet 15 is provided. The additional adhesive layer 90 bonds the barrier sheet 20 and the lower-layer sheet 15.

That is, the adhesive layer 80 and the additional adhesive layer 90 result in the formation of the outer-layer nonwoven fabric 40, the barrier sheet 20, and the lower-layer sheet 15 as one unit.

### (2) Application Pattern of Adhesive

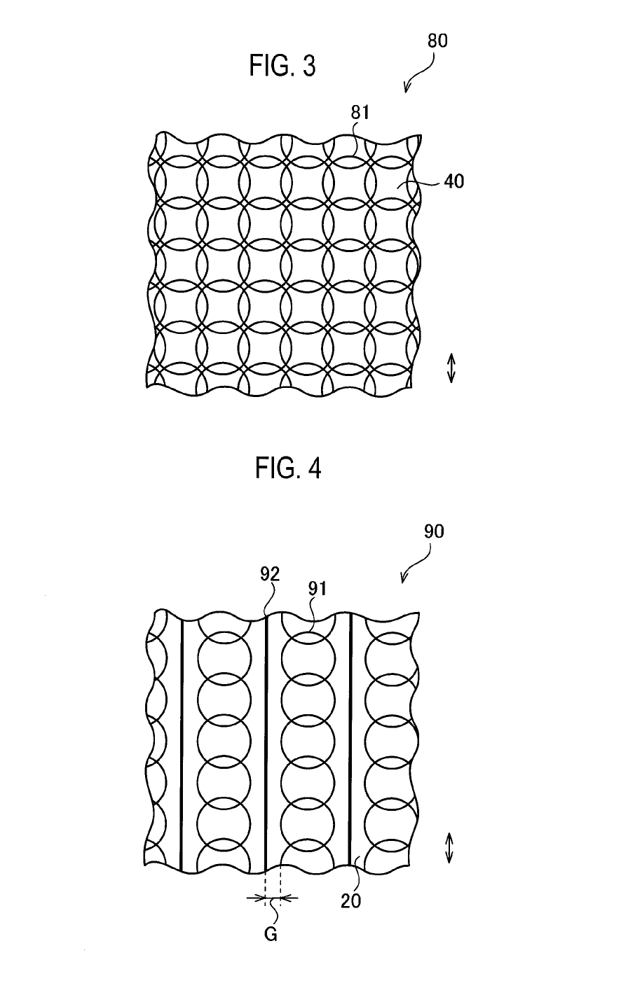

Next, an application pattern of the adhesive in the above-described adhesive layer 80 and the additional adhesive layer 90 will be described. FIG. 3 illustrates an application pattern of the adhesive in the adhesive layer 80. FIG. 4 illustrates an application pattern of the adhesive in the additional adhesive layer 90.

### (2.1) Adhesive Layer 80

As shown in FIG. 3, in the adhesive layer 80, the application pattern of an adhesive 81 has spiral shape. The adhesive 81 forms an application pattern in which the spiral shape is repeated along the longitudinal direction of the absorbent chassis 50.

Furthermore, such an application pattern crosses an orientation (the direction of an arrow in FIG. 1 and FIG. 3) of the fibers forming the outer-layer nonwoven fabric 40 and an orientation of the resin forming the barrier sheet 20. The orientation of the fibers forming the outer-layer nonwoven fabric 40 means a direction in which the fibers forming the outer-layer nonwoven fabric 40 extend, and more specifically, means a direction in which a majority of fibers (including the resin) forming the outer-layer nonwoven fabric 40 extend. Furthermore, the orientation of the resin forming the barrier sheet 20 also carries the same meaning as the outer-layer nonwoven fabric 40. A method of measuring the orientation of fibers and the resin will be described later.

The adhesive 81 is applied so as to cover the entire surface of the barrier sheet 20, and the barrier sheet 20 and the outer-layer nonwoven fabric 40 are fixed by using a surface that is broader than a line and a point. More specifically, the adhesive 81 is applied on the entire surface of the barrier sheet 20 to form a net-like or mesh-like shape, the barrier sheet 20 and the outer-layer nonwoven fabric 40 are bonded and fixed uniformly, and at the same time, the fibers of the outer-layer nonwoven fabric 40 in a non-bonded region have a proper degree of freedom.

Examples of the adhesive 81 include a rubber-type hot-melt adhesive in which a styrene elastomer and EVA are used as a base polymer, and a hot-melt adhesive in which an amorphous poly-alpha olefin is used as a base polymer. Furthermore, the basis weight of the adhesive 81 forming the adhesive layer 80 is preferably 1.0 to 10 g/m². If the basis weight is less than 1.0 g/m², the fiber damage becomes remarkable at the time of engagement and disengagement of the fastening tapes 71 and 72, and therefore, it is not preferable. On the other hand, if the basis weight exceeds 10 g/m², the adhesive 81 restricts the degree of freedom of the fibers due to which the disengagement strength declines, and therefore, it is not preferable.

In addition, the bonding strength of the adhesive layer 80 is preferably between 1.0 and 10 N/25 mm. If the bonding strength is less than 1.0 N/25 mm, the outer-layer nonwoven fabric 40 is pulled by the shearing force of the male members 71a, 72 (hook), and the outer-layer nonwoven fabric 40 and the barrier sheet 20 may peel off. On the other hand, if the bonding strength exceeds 10 N/25 mm, then when the outer-layer nonwoven fabric 40 is pulled by the shearing force of the male members 71a, 72 (hook), the relief of stress (expanding and contrasting of fibers) is not possible, and therefore, the stress is transferred to the barrier sheet 20 due to which the barrier sheet 20 elongates, and perforations, for example, occur easily. A method of measuring the bonding strength will be described later.

It should be noted that the application pattern of the adhesive 81 is not limited to the spiral shape as shown in FIG. 3. Specifically, any application pattern may be acceptable such as that which is formed by coating in a spiral-shaped pattern, coating in an omega (Ω)-shaped pattern, coating in a wave-shaped pattern, or coating in a sheet-shaped pattern, as long as the application pattern crosses the orientation of the fibers forming the outer-layer nonwoven fabric 40, and is also continuous in the longitudinal direction and the shorter direction of the absorbent chassis 50. Coating in a sheet-shaped pattern is a pattern in which a fine thread-like hot-melt adhesive resin is applied on the entire surface of a region to be bonded to result in a sheet-like state. Even in such a case, the application pattern of the adhesive resin is to include an element elongated in the direction that crosses the orientation of the fibers (the orientation of the resin) of the outer-layer nonwoven fabric 40 (the barrier sheet 20), and the element constituting the application pattern is to cross in a mesh-like shape.

That is, the application pattern of the adhesive 81 may be acceptable as long as it is shaped in the form of a net or a mesh, and as a result of such an application pattern, the barrier sheet 20 and the outer-layer nonwoven fabric 40 are fixed uniformly, and at the same time, the fibers of the outer-layer nonwoven fabric 40 positioned in the unbonded region between the fibers have the proper degree of freedom, hence preferable. On the other hand, in a case of coater coating (applying the adhesive thinly over a surface), the adhesive is applied on the entire surface of the outer-layer nonwoven fabric 40, and thus, the degree of freedom of the outer-layer nonwoven fabric 40 is restricted, resulting in a decline in the disengagement strength, hence not preferable.

### (2.2) Additional Adhesive Layer 90

As illustrated in FIG. 4, in the additional adhesive layer 90, the application pattern of an adhesive 91 has spiral shape similarly to the adhesive 81. The adhesive 91 forms an application pattern in which the spiral shape is repeated along the longitudinal direction of the absorbent chassis 50. Furthermore, in the additional adhesive layer 90, the application pattern of an adhesive 92 stretches along the longitudinal direction of the absorbent chassis 50, and specifically, has an approximately parallel linear shape with respect to the longitudinal direction of the absorbent chassis 50.

That is, the additional adhesive layer 90 includes a portion (a first portion) having an application pattern of the adhesive 91 that crosses the orientation (the direction of an arrow shown in FIG. 4) of the resin configuring the barrier sheet 20, and a portion (a second portion) having an application pattern of the adhesive 92 that is approximately parallel to the orientation of the resin.

Furthermore, in the shorter direction that is perpendicular to the longitudinal direction of the absorbent chassis 50, an interval G between the portion (the first portion) having the application pattern of the adhesive 91 and the portion (the second portion) having the application pattern of the adhesive 92 is preferably 20 mm or less. A hot-melt adhesive similar to the adhesive 81 can be used as the adhesive 91 and the adhesive 92.

Similarly to the adhesive 81, the application pattern of the adhesive 91 may be accepted as long as it crosses the orientation of the resin forming the barrier sheet 20, and at the same time, continues in the longitudinal direction and the shorter direction of the absorbent chassis 50. Furthermore, the application pattern of the adhesive 92, for example, can be formed by bead coating.

The basis weight of the adhesive 91 and 92 forming the additional adhesive layer 90 can be configured similarly to that of the adhesive 81. Furthermore, a pasting WET strength of the additional adhesive layer 90 is preferably 0.3 N/25 mm or more. If the pasting WET strength is less than 0.3 N/25 mm, the strength is not sufficient so that the barrier sheet 20, for example, peel off at the time of using the disposable diaper 1.

### (3) Method of Measuring the Orientation of the Fibers (Resin) of the Outer-Layer Nonwoven Fabric 40 (The Barrier Sheet 20)

It is possible to determine the orientation of the fibers of the outer-layer nonwoven fabric 40 and the orientation of the resin of the barrier sheet 20 on the basis of the tensile strength of the sheet (nonwoven fabric) made of thread-like fibers and the sheet (film) made of resin. Specifically, it is possible to measure the orientation of the fibers or the resin can be measured according to a procedure described below.

Create a sample of a sheet (the outer-layer nonwoven fabric 40 or the barrier sheet 20) to be measured that extends along the lengthwise direction and the crosswise direction of the product.

Measure the tensile strength at the time of elongation of the sample by a predetermined amount by using a tensile strength tester.

For the tensile strength test, a tensile strength tester manufactured by Instron Japan Co., Ltd. (for example, model: 5564) was used. A sample having a size (10-mm width and 100-mm length) matching a sample gripping portion of the tester is pulled at a tension rate of 100 mm/min, and the tensile strength is measured when the sample is distorted by 10% with respect to the initial length. In this case, when the tensile strength of the sample extending along a lengthwise direction Y is greater than that of the sample extending along a crosswise direction X, it is possible to determine that the fibers (resin) are oriented along the lengthwise direction Y

### (4) Method of Measuring Stress of the Side Panels 60

It is possible to measure the stress at the time of maximum elongation of the side panels 60 according to a procedure described below.

Separate the side panels 60, including the portion bonded with the absorbent chassis 50, from the absorbent chassis 50.

Cut out the separated side panels 60 at a width of 50 mm, and measure a load (tensile strength) at the time of elongation of the side panels 60 in the expanding and contrasting direction (the shorter direction of the absorbent chassis 50, specifically, the widthwise direction of the disposable diaper 1) by using a tensile strength tester.

For the tensile test, a tensile strength tester manufactured by Instron Japan Co., Ltd. (for example, model: 5564) was used. At this time, fix the bonded ends of the side panels 60 and the absorbent chassis 50, as well as the male member 71a or the male member 72a to the chuck. Following this, the load at the time of maximum elongation of the side panels 60 was measured at a tension rate of 300 mm/min.

### (5) Method of Measuring the Bonding Strength

It is possible to measure the bonding strength between the outer-layer nonwoven fabric 40 and the barrier sheet 20, and that between the barrier sheet 20 and the lower-layer sheet 15 according to a procedure described below.

From the region of the absorbent chassis 50 where the fastening tapes 71 and 72 are affixed, cut out a sample of a size of 25-mm width×100-mm length.

Sandwich the sample by a measurement chuck (at top and bottom two places), move the upper measurement chuck at a direction of 180° with respect to the lower measurement chuck (in a vertical upward direction) at a speed of 100 mm/min, and measure the maximum speed (N) at that time. For the bonding strength test, a tensile strength tester manufactured by Instron Japan Co., Ltd. (for example, model: 5564) was used. It should be noted that the pasting WET strength of the additional adhesive layer 90 was measured by using a similar method.

As described above, in the disposable diaper 1, the barrier sheet 20 and the outer-layer nonwoven fabric 40 are fixed over the surface by the adhesive layer 80. The adhesive layer 80 has an application pattern that crosses the orientation of the fibers forming the outer-layer nonwoven fabric 40 and the orientation of the resin forming the barrier sheet 20, specifically, the adhesive layer 80 has an application pattern in which the spiral shape is repeated along the longitudinal direction of the absorbent chassis 50.

In such disposable diaper 1, even when the male members 71a, 72a of the fastening tapes 71 and 72 are engaged with the outer-layer nonwoven fabric 40 and the fastening tapes 71, 72 are pulled up, a completely opposite force (retaining force) by which the outer-layer nonwoven fabric 40 is kept remaining at the same position is generated against the force for pulling up the fastening tapes 71, 72.

Therefore, the retaining force of the entire region of the outer-layer nonwoven fabric 40 allows for reduction of stretching and undoing of the fibers of the unbonded outer-layer nonwoven fabric 40. That is, it is possible to restrain the detaching of the fibers of the outer-layer nonwoven fabric 40 engaged with the male members 71a, 72a from the male members 71a, 72a before the elongation due to disengagement, and the tearing of the fibers entwined with the male members 71a, 72a. Furthermore, because the application pattern of the adhesive 81 is such that the spiral shape is repeated along the longitudinal direction of the absorbent chassis 50, the adhesive 81 hardly seeps out to the surface of the outer-layer nonwoven fabric.

That is, according to the disposable diaper 1, when using a structure in which rather than making use of a target tape, the fastening tapes 71, 72 are directly engaged with the outer-layer nonwoven fabric 40, it is possible to maintain the engagement force of the fastening tapes 71, 72 even after repeated use while ensuring the strength of the outer-layer nonwoven fabric 40.

In addition, in the present embodiment, the barrier sheet 20 and the lower-layer sheet 15 are bonded by the additional adhesive layer 90. The additional adhesive layer 90 includes a portion having an application pattern of the adhesive 91 that crosses the orientation of the resin configuring the barrier sheet 20, and a portion having an application pattern of the adhesive 92 that is approximately parallel to the orientation of the resin configuring the barrier sheet 20.

Therefore, the outer-layer nonwoven fabric 40, the barrier sheet 20, and the lower-layer sheet 15 are formed as one unit, and the elongation of the barrier sheet 20 is restrained even when a shearing stress is exerted on the outer-layer nonwoven fabric 40, the barrier sheet 20, and the lower-layer sheet 15. When the elongation of the barrier sheet 20 is restrained, the barrier sheets 20 are hardly bonded to each other, and the joining and twisting between the barrier sheets 20, as well as the perforations in the barrier sheet 20 due to such joining and twisting are restrained.

Furthermore, the application pattern of the adhesive 92 is a straight line by bead coating, and thus, the additional adhesive layer 90 can withstand the movement of the wearer even when the additional adhesive layer 90 becomes wet by a bodily waste of the wearer, and the bonding strength by which the barrier sheet 20 and the lower-layer sheet 15 do not peel off can be easily ensured. It should be noted that when the application pattern of the adhesive is non-continuous such as curtain coating, moisture easily enters in between the adhesive, making it difficult to increase the bonding strength.

In addition, in the present embodiment, the interval between the portion having the application pattern of the adhesive 91 and the portion having the application pattern of the adhesive 92 is set to 20 mm or less. Therefore, the additional adhesive layer 90 can withstand the movement of the wearer even when the additional adhesive layer 90 becomes wet by the bodily waste of the wearer, and the bonding strength by which the barrier sheet 20 and the lower-layer sheet 15 do not peel off can be easily ensured.

The present embodiment is designed such that in a state where the side panels 60 are elongated to the maximum extent and the male member 71a and the male member 72a are engaged with the outer-layer nonwoven fabric 40, the stress exerted on the side panels 60 is 4.0 N or less. Therefore, a load caused by the shearing force is hardly exerted on the outer-layer nonwoven fabric 40, which is a region in which the male member 71a and the male member 72a are engaged. In other words, the damage to the outer-layer nonwoven fabric 40 caused by the repeated use of the disposable diaper 1 can be retarded.

In addition, the male member 71a and the male member 72a can be engaged to any region (within a frame indicated by a dashed line in FIG. 1) on the surface of the outer-layer nonwoven fabric 40, and because the side panels 60 have flexibility, the disposable diaper 1 can comply with the most appropriate style of wearing in accordance with various body types and physical conditions of the wearer, particularly elderly people.

### (6) Other Embodiments

As described above, the present invention is disclosed through the embodiments of the present invention. However, it should not be interpreted that the statements and drawings constituting a part of the present disclosure limit the present invention. From this disclosure, a variety of alternate embodiments, examples, and applicable techniques will become apparent to one skilled in the art.

For example, in the above-described embodiment, the side panels 60 had flexibility; however, the side panels 60 need not necessarily have flexibility.

Furthermore, in the above-described embodiment, the barrier sheet 20 and the lower-layer sheet 15 were bonded by the additional adhesive layer 90, however, the barrier sheet 20 and the lower-layer sheet 15 need not necessarily be bonded by the additional adhesive layer 90.

In addition, in the above-described embodiment, the explanation was provided with the disposable diaper 1 as an example, however, the absorbent article to which the present invention can be applied is not limited to a disposable diaper, and can be widely applied in an absorbent article having a male member of a mechanical fastener provided at the sides of the side panels.

As described above, needless to say, the present invention includes various embodiments and the like not described here. Therefore, the technical range of the present invention is to be defined only by the inventive specific matter according to the adequate claims from the above description.

In addition, the entire content of Japanese Patent Application No. 2011-020259 (filed on Feb. 1, 2011) is incorporated in the present specification by reference.

### INDUSTRIAL APPLICABILITY

According to the characteristics of the present invention, it is possible to provide an absorbent article, such as a disposable diaper, by which it is possible to maintain the engagement force of the fastening tape even after repeated use while ensuring the strength of the outer-layer nonwoven fabric, in a case of using a structure in which rather than making use of a target tape, the fastening tape is engaged directly with the outer-layer nonwoven fabric.

### REFERENCE SIGNS LIST

1 . . . Disposable diaper
10 . . . Topsheet
15 . . . Lower-layer sheet
20 . . . barrier sheet
30 . . . Absorber
40 . . . Outer-layer nonwoven fabric
45 . . . LSG nonwoven fabric
50 . . . Absorbent chassis
60 . . . Side panels
71, 72 . . . Fastening tapes
71a, 72a . . . Male members
80 . . . Adhesive layer
90 . . . Additional adhesive layer
81, 91, 92 . . . Adhesive

1. An absorbent article comprising:
a liquid-permeable topsheet, a liquid-impermeable barrier sheet, a liquid-retentive absorber provided between the topsheet and the barrier sheet, and an outer-layer nonwoven fabric provided outside the barrier sheet, comprising, a substantially longitudinally-elongated absorbent chassis;
side panels extending from sides of the absorbent chassis;
a male member of a mechanical fastener provided at sides of the side panels; and an adhesive layer arranged between the barrier sheet and the outer-layer nonwoven fabric, and formed by an adhesive that bonds the barrier sheet and the outer-layer nonwoven fabric, wherein
the adhesive layer has an application pattern of the adhesive, the application pattern crossing an orientation of fibers forming the outer-layer nonwoven fabric and an orientation of a resin forming the barrier sheet.

2. The absorbent article according to claim 1, wherein the application pattern has spiral shape repeated along the longitudinal direction of the absorbent chassis.

3. The absorbent article according to claim 1, further comprising:
a lower-layer sheet provided between the barrier sheet and the absorber, and
an additional adhesive layer formed by an adhesive that bonds the barrier sheet and the lower-layer sheet, wherein
the additional adhesive layer comprises:
a first portion having an application pattern of the adhesive, the pattern crossing the orientation of the resin configuring the barrier sheet, and
a second portion having an application pattern of the adhesive, the pattern being approximately parallel to the orientation of the resin.

4. The absorbent article according to claim 3, wherein an interval between the first portion and the second portion in a shorter direction perpendicular to the longitudinal direction of the absorbent chassis is 20 mm or less.

5. The absorbent article according to claim 3, wherein
the application pattern has spiral shape repeated along the longitudinal direction of the absorbent chassis in the first portion, and has linear shaped extending along the longitudinal direction of the absorbent chassis in the second portion.

6. The absorbent article according to claim 1, wherein the side panels expand and contrast in a shorter direction perpendicular to the longitudinal direction of the absorbent chassis.
